(19) United States
(12) Patent Application Publication
(10) Pub. No.: US 2023/0394992 A1
(43) Pub. Date: Dec. 7, 2023

(54) Olfactory Product Labeling System

(71) Applicant: Anitra Wilson-Lundblad, Reno, NV (US)

(72) Inventor: Anitra Wilson-Lundblad, Reno, NV (US)

(21) Appl. No.: 17/833,899

(22) Filed: Jun. 7, 2022

(51) Int. Cl. G09F 3/00; C11B 9/00

(52) U.S. Cl. CPC G09F 3/0291; C11B 9/00; G09F 2003/0257

(57) ABSTRACT

An olfactory product labeling system is provided which may utilize a base substrate having a first side and an opposing second side. The system may have one or more layers of fragrance coating disposed upon the second side of the base substrate. The one or more layers of fragrance coating may be disposed across the base substrate in the form of a stack. Each layer of fragrance coating may have a plurality of micro-encapsulation elements therein. Further, the system may utilize an abrasion element that is coupled adjacent the base substrate and the one or more layers of fragrance coating.

### BACKGROUND OF THE INVENTION

Almost any tangible product being sold on the market today is sold with an associated product label. The global market for product labeling has been estimated to be around $40 billion annually with more than a trillion total product labels being printed in 2019. Product labels may take many forms including shrink labels, sleeve labels, adhesive labels, pressure-sensitive labels, in-mould labels, dry peel labels, fold out labels, reseal labels and the like.

In most instances, the objectives in using a product label mainly involve communication of information to a prospective consumer. For instance, a product label is designed to give the prospective consumer an idea of what is contained inside of the product packaging. For instance, fruit produce often contains an adhesive sticker labeling the fruit by type, origin and even texture, taste and the like. However, some products are sealed within an opaque container that does not allow the product to be viewed, smelled, tasted or otherwise sampled prior to purchasing.

Further, some products may be sampled but are preferably not to be sampled by the prospective consumer. For example, a candle is often contained within a sealed glass jar having a removable lid. While the lid may be removed to sample the smell of the candle, too much sampling of the candle may lead to premature wear of the product packaging and product label which may act as a deterrent to the product being sold to the prospective consumer. Therefore, it would be advantageous to allow a consumer to sample the product in as many ways as possible without actually accessing the inside of the product packaging.

Other products requiring a constant seal for product preservation such as wine, beer, soda and the like cannot be accessed for sampling. Therefore, it would be advantageous to provide a product label that allows a consumer to sample the taste and smell of such a sealed product without accessing the product content under seal. Further, it would be advantageous to provide such a product label that offers a form factor that does not interfere with the shipping and handling process of a product packaging.

Additionally, existing solutions for product labeling that offer a reusable means of sampling the taste and smell of product contents often are not sanitary. For instance, a scratch and sniff substrate requires a user to physically utilize their fingernails to provide abrasion to the substrate. When utilized iteratively, the scratch and sniff substrate would require a plurality of different sets fo fingernails to be used upon the substrate surface which provides a transmission vector for various pathogens between members of the public population. Therefore, it would be advantageous to provide a product label that allows for sampling of taste and smell of a product in a more sanitary manner than existing solutions. Reducing the risk of pathogen transmission upon the surface of a product label would increase the marketability of the product, particularly in a commercial environment operating within a highly-contagious viral epidemic affecting consumer habits.

### BRIEF DESCRIPTION OF THE DRAWINGS

FIG. 1 is a side cross-sectional view of an olfactory product labeling system having a removable protective layer in accordance with some embodiments of the present invention.

FIG. 2 is a side cross-sectional view of an olfactory product labeling system having an abrasion element in accordance with some embodiments of the present invention.

FIG. 3A is an overview of an olfactory product labeling system disposed upon a product label substrate in accordance with some embodiments of the present invention.

FIG. 3B is a perspective view of an abrasion element of an olfactory product labeling system in accordance with some embodiments of the present invention.

FIG. 4 is an overview of an olfactory product labeling system disposed with an abrasion element upon a product label substrate in accordance with some embodiments of the present invention.

FIG. 5 is an overhead plan view of an olfactory product labeling system comprising an abrasion element in accordance with some embodiments of the present invention.

FIG. 6 is a perspective view of an olfactory product labeling system having a multilayered pad with removable adhesive layers in accordance with some embodiments of the present invention.

### DETAILED DESCRIPTION OF THE INVENTION

Before describing the present invention in detail, it is to be understood that the invention is not limited to any one of the particular embodiments, which of course may vary. It is also to be understood that the terminology used herein is for the purpose of describing particular embodiments only, and therefore is not necessarily intended to be limiting. As used in this specification and the appended claims, terms in the singular and the singular forms “a,” “an,” and “the” include plural referents unless the context clearly dictates otherwise. Thus, for example, reference to “an olfactory product labeling system” also includes a plurality of olfactory product labeling systems and the like.

In some embodiments, an olfactory product labeling system is provided comprising a base substrate comprising a first side and an opposing second side; one or more layers of fragrance coating disposed upon the second side of the base substrate, wherein: the one or more layers of fragrance coating are disposed across the base substrate forming a stack, and each layer of fragrance coating comprises a plurality of micro-encapsulation elements; and an abrasion element coupled adjacent the base substrate and the one or more layers of fragrance coating.

In some embodiments, the base substrate is fabricated from one or more conformable materials manipulated between a concave flexure and a convex flexure.

In some embodiments, the first side of the base substrate comprises a coupling mechanism.

In some embodiments, each of the plurality of micro-encapsulation elements comprise a spherical shell layer having a diameter within the range between 5 microns and 50 microns.

In some embodiments, each of the plurality of micro-encapsulation elements comprise one or more odorants contained therein.

In some embodiments, the plurality of micro-encapsulation elements comprise a first plurality of micro-encapsulation elements each comprising a first odorant disposed therein and a second plurality of micro-encapsulation elements each comprising a second odorant disposed therein, wherein the second odorant is different from the first odorant.

In some embodiments, the abrasion element comprises: a first extended state where the abrasion element is disposed at a first lateral end of the one or more layers of fragrance coating; a second extended state where the abrasion element is disposed at a second lateral end of the one or more layers of fragrance coating; and a transitional state where the abrasion element is disposed between the first and second lateral end of the one or more layers of fragrance coating.

In some embodiments, in the transitional state, the abrasion element applies an abrasive force exceeding a threshold rupturing force of an encapsulating shell of one or more of the plurality of micro-encapsulation elements.

In some embodiments, rupturing the encapsulating shell of any of the plurality of micro-encapsulation elements releases one or more odorants encapsulated therein.

In some embodiments, the threshold rupturing force is within the range between 25 millinewtons and 300 millinewtons.

In some embodiments, the abrasion element comprises a plurality of guide tabs.

In some embodiments, each of the plurality of guide tabs are disposed within respective structural support cavities.

In some embodiments, the structural support cavities comprise a width dimension equal to that of the one or more layers of fragrance coating.

In some embodiments, the abrasion element comprises an abrasive pad having a planar shape with a length dimension equal to that of the one or more layers of fragrance coating.

In some embodiments, the abrasion element comprises an abrasive pad that contacts the one or more layers of fragrance coating upon flexure of the abrasion element.

In some embodiments, the abrasion element comprises an actuation handle.

In some embodiments, the one or more layers of fragrance coating are disposed between a plurality of structural support elements.

In some embodiments, the one or more layers of fragrance coating comprise a removable protective layer disposed over a top surface thereof.

In some embodiments, an olfactory product labeling system is provided comprising a base substrate comprising a first side and an opposing second side; one or more layers of fragrance coating disposed upon the second side of the base substrate, wherein: the one or more layers of fragrance coating are disposed across the base substrate forming a stack, and each layer of fragrance coating comprises a plurality of micro-encapsulation elements; and an abrasion element coupled adjacent the base substrate and the one or more layers of fragrance coating, wherein the abrasion element comprises: a first extended state where the abrasion element is disposed at a first lateral end of the one or more layers of fragrance coating; a second extended state where the abrasion element is disposed at a second lateral end of the one or more layers of fragrance coating; and a transitional state where the abrasion element is disposed between the first and second lateral end of the one or more layers of fragrance coating.

In some embodiments, an olfactory product labeling system is provided comprising a base substrate comprising a first side and an opposing second side; one or more layers of fragrance coating disposed upon the second side of the base substrate, wherein: the one or more layers of fragrance coating are disposed across the base substrate forming a stack, and each layer of fragrance coating comprises a plurality of micro-encapsulation elements, the one or more layers of fragrance coating are disposed between a plurality of structural support elements; and an abrasion element coupled adjacent the base substrate and the one or more layers of fragrance coating, wherein: the abrasion element comprises a plurality of guide tabs each disposed within respective structural support cavities of the structural support elements and an abrasive pad having a planar shape with a length dimension equal to that of the one or more layers of fragrance coating, and the structural support cavities comprise a width dimension equal to that of the one or more layers of fragrance coating.

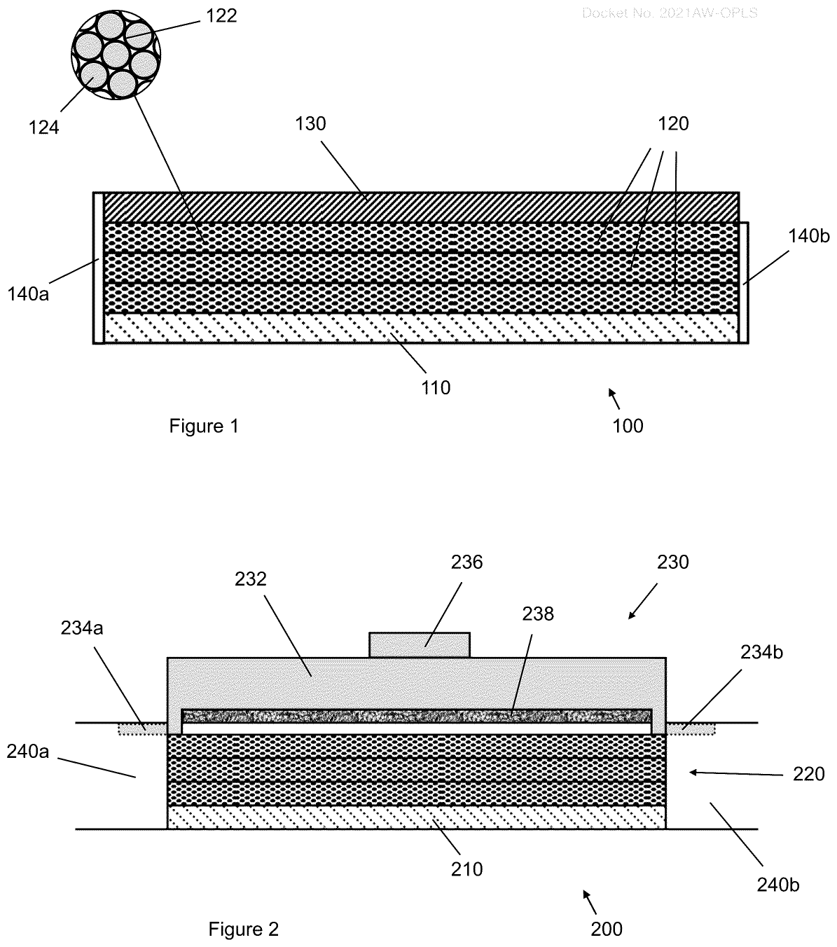

Exemplary embodiments of the present invention are illustrated in the accompanying figures. As shown in FIG. 1, a side cross-sectional view of an olfactory product labeling system 100 having a removable protective layer 130 is provided. The olfactory product labeling system 100 may comprise a base substrate 110, a fragrance coating 120, the removable protective layer 130 and one or more structural support elements 140a, 140b.

The base substrate 110 may comprise a generally thin planar layer having a first side and an opposing second side as shown in FIG. 1. The base substrate 110 itself may be fabricated from one or more conformable materials that allow the base substrate 110 to be manipulated between concave and convex flexures. The concave and convex flexures of the base substrate 110 allow for coupling to any shape or a product packaging surface. The first side of the base substrate 110 may comprise one or more layers of the fragrance coating 120 disposed there across in a similarly planar manner. Additionally, the first side of the base substrate 110 may be situated such that it faces a prospective customer to provide interaction therewith.

The second side of the base substrate 110 may comprise a coupling mechanism disposed thereupon. In some embodiments, the coupling mechanism may take the form of an adhesive layer, a magnetic layer and the like or any combination thereof. Further, the second side of the base substrate 110 may be disposed adjacent a product packaging surface (not shown) and secured thereto by utilization of the coupling mechanism.

The fragrance coating 120 may comprise one or more layers of fragrance coatings that may be disposed upon the first side of the base substrate 110 in the form of a multilayered stack as shown in FIG. 1 and related figures. Utilization of a plurality of layers of fragrance coating may be advantageous in providing prolonged use of the olfactory product labeling system 100. For instance, typical abrasion-sensitive scent releasing elements experience abrasion in a common area along their surfaces, thereby reducing the effectiveness of the scent releasing elements when that common area has experienced too much abrasion.

Advantageously, using a plurality of layers of fragrance coating 120 allows the olfactory product labeling system 100 to be utilized even after an outermost layer of the fragrance coating 120 has been worn through in the common area from abrasion by a prospective customer sampling the scent of a product contents. While the edges of the outermost layer may still be intact, many prospective customers only utilize the common area (e.g. the central portion) and are less likely to utilize the edges of the fragrance coating 120 layers. While three layers of fragrance coating 120 are illustrated in FIG. 1, any similar number of layers of fragrance coating 120 may be utilized in constituting the fragrance coating 120.

Further, each of the one or more layers of fragrance coating 120 may be constituted by a plurality of spherical micro-encapsulation elements 122 arranged in a patterned or non-pattered array as shown in FIG. 1. Each of the plurality of spherical micro-encapsulation elements 122 may be disposed adjacent one another or may be suspended within a solid suspension material. Moreover, each of the plurality of spherical micro-encapsulation elements 122 may be filled with one or more odorants 124 which may be encapsulated by one or more shell layers.

Specifically, the one or more shell layers of each of the plurality of spherical micro-encapsulation elements 122 may be pierced by physical abrasive force applied by a prospective customer (via an abrasion element in FIG. 2) in order to release the one or more odorants 124 contained therein. The abrasive force required to pierce the plurality of spherical micro-encapsulation elements 122 should be no greater than casual force applied by an average customer in the marketplace. Similarly, the plurality of spherical micro-encapsulation elements 122 that are not adjacent the abrasive force should collectively be structurally robust enough in the patterned or non-patterned array to withstand the distributed abrasive force applied to the exposed surface of the fragrance coating 120. Upon piercing of the spherical micro-encapsulation elements 122, the one or more odorants 124 may diffuse in an airborne manner in all directions such that the prospective customer is able to sample the smell and/or taste of the contents contained within the product packaging.

Generally, in this and other embodiments, the shell structure of each of the spherical micro-encapsulation elements 122 may be robust up to a predetermined threshold rupturing force that is sufficient to compromise the structural integrity of the plurality of spherical micro-encapsulation elements 122 that experience the abrasive force applied thereto. In some embodiments, the threshold rupturing force may be between 25 millinewtons and 300 millinewtons. A threshold rupturing force below 25 millinewtons would render the micro-encapsulation elements 122 too easily ruptured thereby rendering the fragrance coating 120 too fragile and the olfactory product labeling system 100 non-functional. A threshold rupturing force above 300 millinewtons would render the micro-encapsulation elements 122 too difficult to rupture thereby rendering the fragrance coating 120 too robust and the olfactory product labeling system 100 non-functional. Upon rupturing of each micro-encapsulation element 122, a predetermined amount of odorant 124 is released therefrom and diffuses into the environmental air within sense-proximity of the prospective customer.

Further, it is advantageous that the type of odorants 124 utilized within the micro-encapsulation elements 122 of the fragrance coating 120 correspond to the inherent scent and/or taste of the product contained within the product packaging. For instance, a candle comprising a lemon and eucalyptus scent may comprise both lemon and eucalyptus odorants 124 contained within the micro-encapsulation elements 122 of the olfactory product labeling system 100 applied to the candle product packaging. Moreover, in this instance, each micro-encapsulation element 122 may comprise both lemon and eucalyptus odorants 124 or separate micro-encapsulation elements 122 may respectively comprise lemon or eucalyptus odorants 124 which thereby provide a combined scent upon abrasion by a prospective customer. Further, in such an instance, each layer of fragrance coating 120 may comprise only lemon odorants 124 or only eucalyptus odorants 124 such that, when the series of layers of fragrance coating 120 are worn by abrasion, a combination of lemon and eucalyptus odorants 124 are released from respective micro-encapsulation elements 122.

With respect to the plurality of spherical micro-encapsulation elements 122, each may comprise a spherical enclosure on the micron diameter scale having one or more aroma-generating compounds, such as odorants 124, contained therein. Specifically, each spherical enclosure may be fabricated from gelatin or plastic polymer materials and may comprise a diameter within a range between 5 microns and 50 microns depending upon the type of odorants 124 utilized and the form factor of the olfactory product labeling system 100 required for the given product packaging it is being applied to.

The removable protective layer 130 may comprise a planar structure that may be disposed directly over the one or layers of fragrance coating 120 in order to protect the contents thereof from abrasion. Further, the removable protective layer 130 may be peeled back from the surface of the fragrance coating 120 layers in order to expose them to the prospective customer. After accessing the fragrance coating 120 and releasing grip of the protective layer 130, the removable protective layer 130 may reflexively recoil back into its position over the surface of the fragrance coating 120.

Advantageously, the prospective customer may forget to replace the protective layer 130 back into place and so the reflexive recoiling of the protective layer 130 ensures that the fragrance coating 120 continues to be protected from abrasion. The protective layer 130 is capable of reflexively recoiling back into position at least in part via the one or more structural support elements 140a, 140b. Specifically, the structural support elements 140a, 140b may aid in maintaining the structure of the olfactory product labeling system 100 before, during and after abrasion by the prospective customer. Further, one of the structural support elements 140a, 140b may robustly couple to the protective layer 130 at a first end thereof to allow the prospective customer to peel it back at a second end thereof.

As shown in FIG. 2, a side cross-sectional view of an olfactory product labeling system 200 having an abrasion element 230 is provided. The olfactory product labeling system 200 may comprise a base substrate 210, one or more layers of fragrance coating 220, the abrasion element 230 and one or more structural support elements 240a, 240b.

The base substrate 210 may comprise a generally thin planar layer having a first side and an opposing second side as shown in FIG. 2. Each of the first side and the opposing second side may be structurally and functionally similar to the base substrate 110 described with respect to FIG. 1. Therefore, the first side of the base substrate 210 may comprise one or more layers of the fragrance coating 220 disposed there across in a similarly planar manner and may further support the abrasion element 230 thereupon. Further, the second side of the base substrate 110 may comprise a coupling mechanism disposed thereupon which may take the form of an adhesive layer, a magnetic layer and the like or any combination thereof.

With specific regard to the base substrate 210 of FIG. 2, the one or more structural support elements 240a, 240b may be structurally, materially and/or functionally identical to the base substrate 210. Specifically, the base substrate 210 and structural support elements 240a, 240b may be fabricated unitary with one another and may form a part of the product packaging or the product label substrate. That is to say that the olfactory product labeling system 200 may be structurally integrated with the product label except for the abrasion element 230 which will be described in greater detail herein.

The fragrance coating 220 may comprise one or more layers of fragrance coatings that may be disposed upon the first side of the base substrate 210 as shown in FIG. 2. Each of the one or more layers of fragrance coatings 220 may be structurally, materially and functionally similar to the one or more layers of fragrance coatings 120 described with respect to FIG. 1. Utilization of a plurality of layers of fragrance coating may be advantageous in providing prolonged use of the olfactory product labeling system 200.

Moreover, the abrasion element 230 of FIG. 2 may further provide the olfactory product labeling system 200 with prolonged use over existing solutions. Specifically, the abrasion element 230 may comprise a planar protective support 232 that serves to protect the one or more layers of fragrance coating 220 from external environmental abrasion during fabrication and/or shipping of the olfactory product labeling system 200. Additionally, the planar protective support 232 provides structural support for the plurality of guide tabs 234a, 234b, the actuation handle 236 and the abrasion pad 238.

The plurality of guide tabs 234a, 234b may be at least partially inserted into structurally corresponding cavities formed within respective structural support elements 240a, 240b. Each of the plurality of guide tabs 234a, 234b may extend from terminal edges of the planar protective support 232 into the cavities of the structural support elements 240a, 240b. In use, the guide tabs 234a, 234b may be utilized to keep the abrasion element 230 within a linear path as a prospective customer is manipulating the abrasion element 230 across the surface of the one or more layers of fragrance coating 220 in a direction that is orthogonal to the vertical and horizontal plane of FIG. 2 (i.e. a z-axis that projects into and out of the page of FIG. 2).

In this and other embodiments, the abrasion element 230 may be manipulated across the one or more layers of fragrance coating 220 starting in a first extended state where the abrasion element 230 is disposed at a first lateral end of the one or more layers of fragrance coating 220, then moving to a transitional state where the abrasion element 230 is disposed between the first and second lateral end of the one or more layers of fragrance coating 220, and finally arriving at a second extended state where the abrasion element 230 is disposed at a second lateral end of the one or more layers of fragrance coating 220. The abrasion element 230 may iteratively move between each of the first extended state, the second extended state and the transitional state.

The actuation handle 236 may be utilized by the prospective customer to grip the abrasion element 230 for ease of use in manipulating the abrasion element 230 across the surface of the one or more fragrance coatings 220. In use, the prospective customer may grip the actuation handle 236 and apply downward force so that the abrasion pad 238 makes physical contact with the outermost surface of the fragrance coating 220 and thereby applies an abrasive force thereto for piercing one or more micro-encapsulation elements therein.

Advantageously, the planar protective support 232 and the plurality of guide tabs 234a, 234b may comprise a shape and be fabricated from one or more materials that allow for vertical flexure of the planar protective support 232 upon the prospective customer applying vertically downward force upon the fragrance coating 220 via the actuation handle 236 and abrasive pad 238. Therefore, as the one or more layers of fragrance coating 220 become worn down via abrasive force, additional prospective customers may apply greater vertically downward forces to the planar protective support 232 and guide tabs 234a, 234b to vertically flex them even further so that the abrasive element 238 may physically contact the fragrance coating 220 even in a worn state where the outermost layer of fragrance coating 220 has been worn away and additional layers of fragrance coating 220 there beneath are now exposed to the abrasive pad 238.

If the guide tabs 234a, 234b are to remain vertically stationary within the cavities of the structural support elements 240a, 240b, then a generally concave wear pattern will be worn into the fragrance coating 220. This may be advantageous in that the central portion of the fragrance coating 220 will be worn down to the base substrate 210 first, thereby allowing visual confirmation that the fragrance coating 220 is nearly fully consumed and a quantitative estimate of the amount of sampling given to the associated product bearing the olfactory product labeling system 200 by prospective customers.

Alternatively, the guide tabs 234a, 234b may be allowed to vertically traverse the cavities formed within the structural support elements 240a, 240b which would allow for an even wear pattern to be formed across the surface of the fragrance coating, thereby prolonging the lifespan of the olfactory product labeling system 200. Additionally, the abrasive pad 238 may extend across the bottom surface of each guide tab 234a, 234b so that the guide tabs may also provide abrasive force to the fragrance coating 220 and thereby further provide a more even wear pattern across the external surface of the fragrance coating 220.

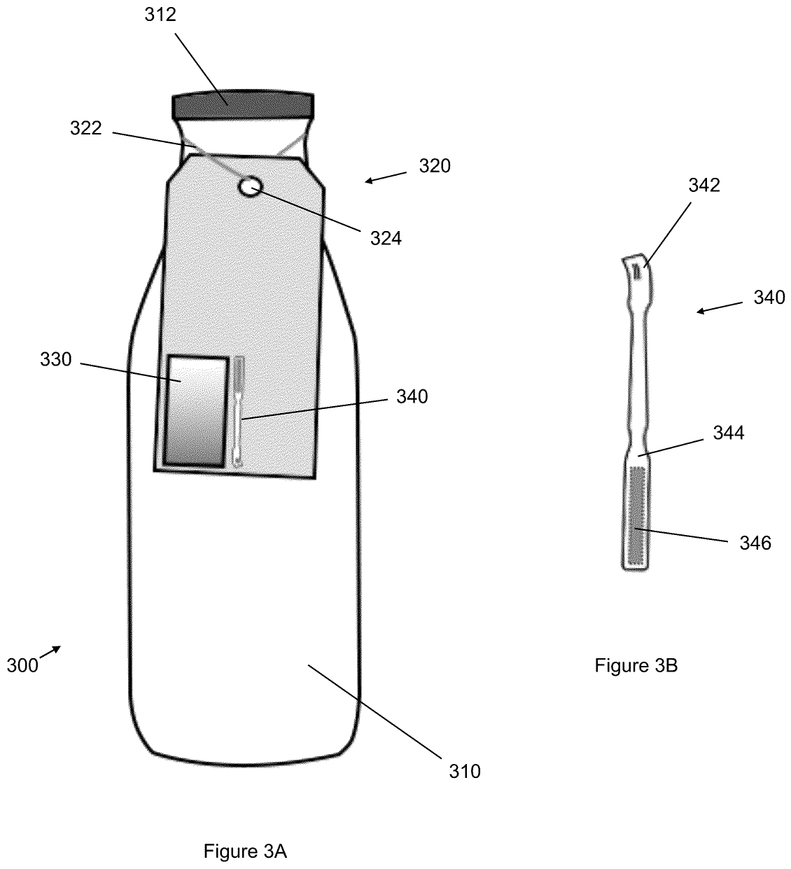

As shown in FIG. 3A, an overview of an olfactory product labeling system 300 disposed upon a product label substrate 320 is provided. The olfactory product labeling system 300 may comprise a product packaging 310, the product label substrate 320, a fragrance coating portion 330 and an abrasive element 340.

The product packaging 310 may comprise a bottle as shown in FIG. 3A or may comprise any other similar packaging such as, but not limited to, carton, box, paper, cardboard, plastic container, glass container and the like or any combination thereof. In this embodiment, the product packaging 310 comprises a sealing element 312 that seals product contents within the product packaging 310 such that a prospective customer cannot smell or taste the product contents prior to purchasing the product. This creates a problem in the market in that the prospective customer does not have reasonable expectations as to the smell and/or taste of the product prior to purchasing of the product.

Advantageously, the product label substrate 320 coupled to the product packaging 310 via a retention element 322 and retention aperture 324 may solve this problem in the market. Specifically, the product label substrate 320 may comprise the fragrance coating portion 330 fabricated thereon in a manner that may be similar to that described with respect to FIGS. 1 and 2. The prospective customer may utilize the abrasive element 340 to apply an abrasive force to the fragrance coating portion 330 in order to pierce one or more spherical micro-encapsulation elements disposed therein to release one or more odorants that provide a preview of the taste and/or smell of the product contents contained within the product packaging 310 without breaking the seal of the sealing element 312.

This solution provides increased reasonable expectations for the prospective customer as to the taste and/or smell of the product content without jeopardizing the shelf-life of the product by breaking the sealing element 312. This provides increased sales to the producers of the product by providing a greater sense of purchasing confidence to the prospective customer. Further, products bearing an olfactory product labeling system 300 may comprise a competitive advantage over products that do not bear an olfactory product labeling system 300 given that a prospective customer may prefer purchasing a product that they would rate at an average score of 7 out of 10 in overall satisfaction based upon the taste and/or smell of the olfactory product labeling system 300 over a product that does not bear an olfactory product labeling system 300 at all and therefore bears a higher risk of having a lower overall satisfaction score after purchasing the product which then cannot be returned.

In some embodiments, the abrasive element 340 may be iteratively attached to and removed from the product label substrate 320 by the prospective customer for repeated use of the olfactory product labeling system 300. Specifically, with reference to FIG. 3B, the abrasive element 340 may comprise an abrasion edge 342, a handle portion 344 and a coupling mechanism 346. The abrasion edge 342 may be utilized to apply abrasive force to the fragrance coating portion 330. The handle portion 344 may be utilized by the prospective customer to leverage the abrasive force to the abrasion edge 342. The coupling mechanism 346 may be utilized to attach the abrasive element 340 to the product label substrate 320. The coupling mechanism 346 may take the form of an adhesive pad, a magnetic strip, a patch of hook and loop fasteners and the like or any combination thereof.

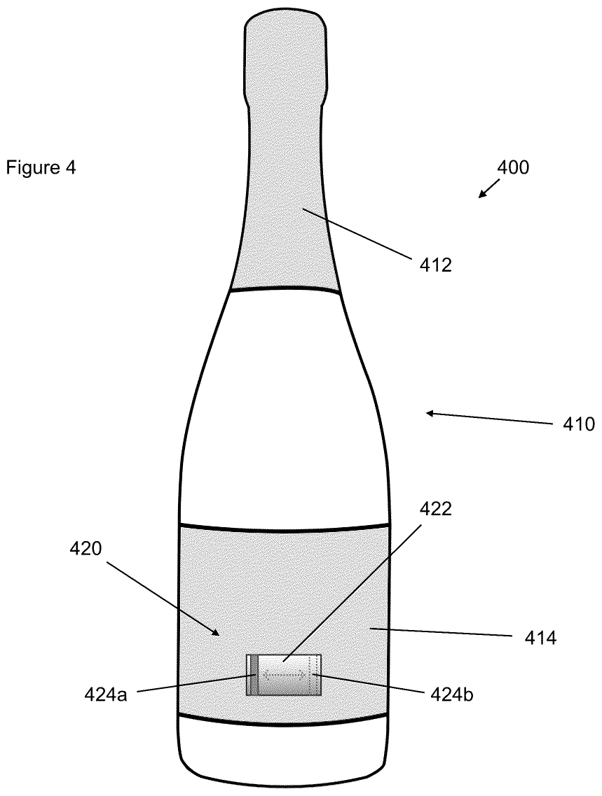

As shown in FIG. 4, an overview of an olfactory product labeling system 400 disposed with an abrasion element 420 upon a product label substrate 414 is provided. The olfactory product labeling system 400 may comprise a product packaging 410 and the abrasion element 420.

The product packaging 410 may comprise an oblong shape typically difficult to apply product labeling thereto. Specifically, the product packaging 410 may comprise a neck portion product label substrate 412 and a base portion product label substrate 414 in the shape of a typical wine bottle as shown in FIG. 4. The neck portion product label substrate 412 may seal the product packaging 410 contents therein. The abrasion element 420 may be applied to either the neck portion product label substrate 412 or the base portion product label substrate 414.

Advantageously, the abrasion element 420 may comprise a flexible nature that allows it to be applied to a diversity of product packaging surface shapes. Specifically, each of the components of the abrasion element 420 may be fabricated from materials that allow vertical (i.e. a z-axis into and out of the page in FIG. 4) flexure thereof to conform to the oblong product surface shapes of the product packaging 410 illustrated in FIG. 4.

Further, the abrasion element 420 may be utilized by a prospective customer to release odorants from a fragrance coating 422 in order to sample the contents contained within the sealed product packaging 410. Specifically, the abrasion element 420 may be similar to the abrasion element 230 of FIG. 2 in that it may traverse across the surface of the fragrance coating 422 from a first position 424a to a second position 424b. The movement of the abrasion element 420 across the fragrance coating 422 provides abrasive force thereto and causes the odorants to be released therefrom.

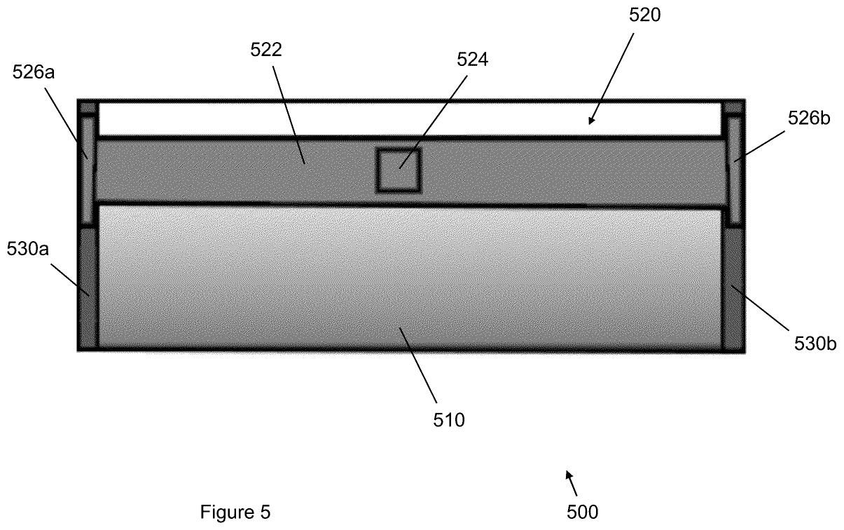

As shown in FIG. 5, an overhead plan view of an olfactory product labeling system 500 comprising an abrasion element 520 is provided. The olfactory product labeling system 500 may comprise a fragrance coating 510, the abrasion element 520 and one or more structural support cavities 530a, 530b.

The fragrance coating 510 may be disposed over a base substrate (not shown) that may couple to one or more structural support elements (not shown) that form the structural support cavities 530a, 530b. The fragrance coating 510 may have abrasive force applied thereto by movement of the abrasion element 520 there across. Such movement may release one or more odorants from at least one of a plurality of spherical micro-encapsulation elements contained within the layer of fragrance coating 510.

The abrasion element 520 may comprise a planar protective support 522 which protects the fragrance coating 510 from external abrasion and supports an actuation handle 524 and a plurality of guide tabs 526a, 526b. Advantageously, the planar protective support 522 and the plurality of guide tabs 526a, 526b may comprise a shape and be fabricated from one or more materials that allow for vertical flexure of the planar protective support 522 upon a prospective customer applying vertically downward force upon the fragrance coating 510 via the actuation handle 524 and an abrasive pad (not shown).

The guide tabs 526a, 526b may be removably disposed within respective structural support cavities 530a, 530b which allow the guide tabs 526a, 526b to be translated along the length of the structural support cavities 530a, 530b as the prospective customer manipulates the abrasion element 520 across the surface of the fragrance coating 510 via the actuation handle 524. In this and other embodiments, the structural support cavities 530a, 530b may comprise a width dimension equal to that of the one or more layers of fragrance coating 510. This is advantageous to allow the abrasion element to be guided across the entire width of the fragrance coating 510 surface so as to release odorants evenly therefrom. Further, the abrasion element 520 may comprise an abrasive pad having a planar shape with a length dimension equal to that of the one or more layers of fragrance coating 510. This is advantageous to allow the abrasive pad of the abrasion element 520 to be physically incident upon the entire length dimension of the fragrance coating 510 surface so as to release odorants evenly therefrom.

Advantageously, the abrasion element 520 may be selectively removed from the remainder of the olfactory product labeling system 500 and reattached thereto in an iterative manner as desired. This allows for a product requiring tight shipping spaces to fit within their shipping container dimensions without having the abrasion element 520 of the olfactory product labeling system 500 protrude therefrom. After shipping but before product shelving, the abrasion element 520 may easily be reattached the remainder of the olfactory product labeling system 500 by reinserting the guide tabs 526a, 526b into the structural support cavities 530a, 530b.

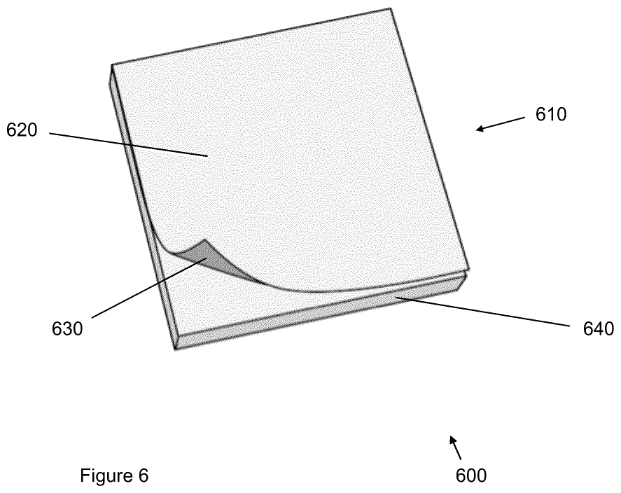

As shown in FIG. 6, a perspective view of an olfactory product labeling system 600 comprising a multilayered pad 610 with a plurality of removable adhesive layers 620 in accordance with some embodiments of the present invention. The multilayered pad 610 of the olfactory product labeling system 600 may comprise may comprise the plurality of removable adhesive layers 620 arranged serially upon one another in a vertical stack 640 as shown in FIG. 6.

Each of the removable adhesive layers 620 may comprise a first side facing outwards and a second side facing inwards towards the remainder of the vertical stack of adhesive layers 620. The first side of each adhesive layer 620 may comprise a protective coating that is robust to adhesion when another adhesive layer 620 is disposed thereover. Further, the protective coating may be robust to external abrasion when the adhesive layer 620 disposed thereover is removed, thus exposing the first side to risk of external abrasion during the shipment and handling of an associated product.

Moreover, the first side is designed to be protective of the second side of the adhesive layer 620 which may comprise an adhesive odorant material 630 that itself may comprise one or more adhesive materials, one or more odorant materials, one or more micro-encapsulation elements, one or more layers of fragrance coating or any combination thereof. Specifically, the first side of each adhesive layer 620 protects the shelf-life and use-life of the adhesive odorant material 630 so that a user may remove one of the adhesive layers 620 and experience the scent of the adhesive odorant material 630 disposed on the second side. The scent of the adhesive odorant material 630 may correspond to product contents contained within a product packaging upon which the multilayered pad 610 of the olfactory product labeling system 600 is to be disposed. In this configuration, the product packaging surface (not shown) may act as a base substrate upon which the vertical stack 640 is to be arranged. Alternatively, a separate base substrate containing the vertical stack 640 may be utilized for adhesive application to the product packaging surface.

In the embodiment of the olfactory product labeling system 600 of FIG. 6, the first side of an adjacent adhesive layer 620 may act as an abrasion element for the adhesive odorant material 630. Specifically, when the user removes an adhesive layer 620 from the multilayered pad 610, the act of adhesive removal of the second side of an adhesive layer 620 from the first side of an adjacent adhesive layer 620 may cause one or more micro-encapsulation elements of the adhesive odorant material 630 to be perforated which releases the scented odorant material therein for the user to experience.

Throughout this disclosure, the phrase ‘modularly coupled’ and similar terms and phrases are intended to convey that any element of a given class of elements may be coupled to another given element and vice versa with equal effect. For example, any extension cord of a plurality of extension cords may be modularly coupled to another extension cord and vice versa with equal effect. Further, throughout this disclosure, the phrase ‘removably coupled’ and similar terms and phrases are intended to convey that a given element may be iteratively coupled to and removed from another given element as desired. For example, a male plug of a first extension cord may be removably coupled to a female plug of a second extension cord as desired.

The specification and drawings are to be regarded in an illustrative rather than a restrictive sense. However, it will be evident that various modifications and changes may be made thereunto without departing from the broader spirit and scope of the invention as set forth in the claims. Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a,” “an,” “the,” and similar referents in the context of describing the disclosed embodiments (especially in the context of the following claims) are to be construed to cover both the singular and the plural, unless otherwise indicated or clearly contradicted by context. The terms “comprising,” “having,” “including,” and “containing” are to be construed as open-ended terms (i.e., meaning “including, but not limited to,”) unless otherwise noted. The term “connected,” where unmodified and referring to physical connections, is to be construed as partly or wholly contained within, attached to, or joined together, even if there is something intervening. Recitation of ranges of values are merely intended to serve as a shorthand method of referring individually to each separate value falling within the range, unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Conjunctive language, such as phrases of the form “at least one of A, B, and C,” or “at least one of A, B and C,” is understood with the context as used in general to present that an item, term, etc., may be either A or B or C, or any nonempty subset of the set of A and B and C, unless specifically stated otherwise or otherwise clearly contradicted by context. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, the term “plurality” indicates a state of being plural (e.g., “a plurality of items” indicates multiple items). The number of items in a plurality is at least two, but can be more when so indicated either explicitly or by context.

The use of any examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described, including the best mode known to the inventors for carrying out the invention. Variations of those embodiments may become apparent to those of ordinary skill in the art upon reading the foregoing description. The inventors expect skilled artisans to employ such variations as appropriate and the inventors intend for embodiments of the present disclosure to be practiced otherwise than as specifically described. Accordingly, the scope of the present disclosure includes all modifications and equivalents of the subject matter recited in the claims appended hereto as permitted by applicable law. Moreover, although above-described elements may be described in the context of certain embodiments of the specification, unless stated otherwise or otherwise clear from context, these elements are not mutually exclusive to only those embodiments in which they are described; any combination of the above-described elements in all possible variations thereof is encompassed by the scope of the present disclosure unless otherwise indicated or otherwise clearly contradicted by context.

All references, including publications, patent applications, and patents, cited are hereby incorporated by reference to the same extent as if each reference were individually and specifically indicated to be incorporated by reference and were set forth in its entirety.

1. An olfactory product labeling system, comprising:
a base substrate comprising a first side and an opposing second side;
one or more layers of fragrance coating disposed upon the second side of the base substrate, wherein:
the one or more layers of fragrance coating are disposed across the base substrate forming a stack, and
each layer of fragrance coating comprises a plurality of micro-encapsulation elements; and
an abrasion element coupled adjacent the base substrate and the one or more layers of fragrance coating.

2. The olfactory product labeling system of claim 1, wherein the base substrate is fabricated from one or more conformable materials manipulated between a concave flexure and a convex flexure.

3. The olfactory product labeling system of claim 1, wherein the first side of the base substrate comprises a coupling mechanism.

4. The olfactory product labeling system of claim 1, wherein each of the plurality of micro-encapsulation elements comprise a spherical shell layer having a diameter within the range between 5 microns and 50 microns.

5. The olfactory product labeling system of claim 1, wherein each of the plurality of micro-encapsulation elements comprise one or more odorants contained therein.

6. The olfactory product labeling system of claim 1, wherein the plurality of micro-encapsulation elements comprise a first plurality of micro-encapsulation elements each comprising a first odorant disposed therein and a second plurality of micro-encapsulation elements each comprising a second odorant disposed therein, wherein the second odorant is different from the first odorant.

7. The olfactory product labeling system of claim 1, wherein the abrasion element comprises:
a first extended state where the abrasion element is disposed at a first lateral end of the one or more layers of fragrance coating;
a second extended state where the abrasion element is disposed at a second lateral end of the one or more layers of fragrance coating; and
a transitional state where the abrasion element is disposed between the first and second lateral end of the one or more layers of fragrance coating.

8. The olfactory product labeling system of claim 7, wherein, in the transitional state, the abrasion element applies an abrasive force exceeding a threshold rupturing force of an encapsulating shell of one or more of the plurality of micro-encapsulation elements.

9. The olfactory product labeling system of claim 8, wherein rupturing the encapsulating shell of any of the plurality of micro-encapsulation elements releases one or more odorants encapsulated therein.

10. The olfactory product labeling system of claim 8, wherein the threshold rupturing force is within the range between 25 millinewtons and 300 millinewtons.

11. The olfactory product labeling system of claim 1, wherein the abrasion element comprises a plurality of guide tabs.

12. The olfactory product labeling system of claim 11, wherein each of the plurality of guide tabs are disposed within respective structural support cavities.

13. The olfactory product labeling system of claim 12, wherein the structural support cavities comprise a width dimension equal to that of the one or more layers of fragrance coating.

14. The olfactory product labeling system of claim 1, wherein the abrasion element comprises an abrasive pad having a planar shape with a length dimension equal to that of the one or more layers of fragrance coating.

15. The olfactory product labeling system of claim 1, wherein the abrasion element comprises an abrasive pad that contacts the one or more layers of fragrance coating upon flexure of the abrasion element.

16. The olfactory product labeling system of claim 1, wherein the abrasion element comprises an actuation handle.

17. The olfactory product labeling system of claim 1, wherein the one or more layers of fragrance coating are disposed between a plurality of structural support elements.

18. The olfactory product labeling system of claim 1, wherein the one or more layers of fragrance coating comprise a removable protective layer disposed over a top surface thereof.

19. An olfactory product labeling system, comprising:
a base substrate comprising a first side and an opposing second side;
one or more layers of fragrance coating disposed upon the second side of the base substrate, wherein:
the one or more layers of fragrance coating are disposed across the base substrate forming a stack, and
each layer of fragrance coating comprises a plurality of micro-encapsulation elements; and
an abrasion element coupled adjacent the base substrate and the one or more layers of fragrance coating, wherein the abrasion element comprises:
a first extended state where the abrasion element is disposed at a first lateral end of the one or more layers of fragrance coating;
a second extended state where the abrasion element is disposed at a second lateral end of the one or more layers of fragrance coating; and
a transitional state where the abrasion element is disposed between the first and second lateral end of the one or more layers of fragrance coating.

20. An olfactory product labeling system, comprising:
a base substrate comprising a first side and an opposing second side;
one or more layers of fragrance coating disposed upon the second side of the base substrate, wherein:
the one or more layers of fragrance coating are disposed across the base substrate forming a stack, and
each layer of fragrance coating comprises a plurality of micro-encapsulation elements,
the one or more layers of fragrance coating are disposed between a plurality of structural support elements; and
an abrasion element coupled adjacent the base substrate and the one or more layers of fragrance coating, wherein:
the abrasion element comprises a plurality of guide tabs each disposed within respective structural support cavities of the structural support elements and an abrasive pad having a planar shape with a length dimension equal to that of the one or more layers of fragrance coating, and the structural support cavities comprise a width dimension equal to that of the one or more layers of fragrance coating.
